(19) United States
(12) Patent Application Publication
(10) Pub. No.: US 2025/0339390 A1
(43) Pub. Date: Nov. 6, 2025

(54) KIT FOR PREPARING INJECTABLE CARMUSTINE SOLUTIONS

(71) Applicant: VIVUS LLC, Campbell, CA (US)

(72) Inventors: Deepak Pragjibhai GONDALIYA, Pune (IN); Hiren Pravinbhai PATEL, Pune (IN); Haresh Ishwarbhai PATEL, Pune (IN); Mukund Keshav GURJAR, Pune (IN)

(21) Appl. No.: 19/267,740

(22) Filed: Jul. 14, 2025

Related U.S. Application Data
(63) Continuation of application No. 17/014,604, filed on Sep. 8, 2020.
Application No. 17/014,604 is a continuation-in-part of application No. 16/584,371, filed on Sep. 26, 2019, now abandoned.

(30) Foreign Application Priority Data
Oct. 4, 2018 (IN) 201821037526

(51) Int. Cl. A61K 31/17; A61K 47/02; A61K 47/10

(52) U.S. Cl. CPC A61K 31/17; A61K 47/02; A61K 47/10

(57) ABSTRACT

The present invention relates to an improved kit for preparing injectable carmustine solutions, methods of preparing and administering such solutions, and methods of treatment with such solutions. The kit includes a product vial containing lyophilised carmustine and a diluent vial containing an ethanol-free non-aqueous diluent. Reconstitution of the lyophilised carmustine in the ethanol-free non-aqueous diluent results in a carmustine solution with improved solubility and stability.

The present application is a continuation of U.S. patent application Ser. No. 17/014,604, filed Sep. 8, 2020, which is a continuation-in-part of U.S. patent application Ser. No. 16/584,371, filed Sep. 26, 2019, which claims the benefit of Indian Patent Application number 201821037526, Oct. 4, 2018, the entire contents of each of which are hereby incorporated by reference.

### FIELD OF THE INVENTION

The present invention relates to an improved kit for preparing injectable carmustine solutions, methods of preparing and administering such solutions, and methods of treatment with such solutions.

### BACKGROUND OF THE INVENTION

Carmustine (bischloroethyl nitrosurea also known as BCNU) is a nitrosurea drug for the treatment of brain cancers owing to its ability to cross the blood-brain barrier and excellent activity against brain tumours.

Carmustine chemically known as 1,3-bis(2-chloroethyl)-1-nitrosourea (shown below) alkylates DNA, RNA and interferes with its synthesis and functions. It also binds and modifies (carbamoylates) glutathione reductase, which consequently leads to cell death.

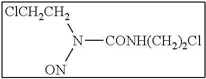

Carmustine is poorly soluble in water and is unstable in many formulations. For instance, carmustine gets readily hydrolyzed in water at pH >6. The solubility and stability issues of carmustine have been discussed previously. See, for example, Levin et al., Selective Cancer Therapeutics, 1989, 5 (1), 33-35.

Carmustine is commercially available as a lyophilized 100 mg powder for injection under the trade name BiCNU® in single dose vials. See the March 2017 prescribing information for BICNU®, which is hereby incorporated by reference. Ethanol (dehydrated alcohol) (3 mL) is co-packaged with the drug product as a sterile diluent for reconstitution. To prepare the drug for administration, three preparation steps need to be performed. First, the lyophilized carmustine is dissolved with the co-packed sterile dehydrated alcohol (3 mL) diluent. Second, the solution is further diluted with 27 mL of sterile water to form the reconstituted solution. Third, the reconstituted solution is further diluted with 5% Dextrose Injection, USP or Sodium Chloride Injection, USP (0.9% sodium chloride). This complicated preparation of carmustine solutions is time-consuming and can to lead to errors in preparation and dosing.

Furthermore, ethanol in injectable products may cause undesirable adverse events, including infusion toxicity and hypersensitivity reactions. The U.S. Food and Drug Administration (FDA) in June 2014 issued a warning that the cancer drug docetaxel may cause symptoms of alcohol intoxication after treatment. See https://www.fda.gov/drugs/drug-safety-and-availability/fda-drug-safety-communication-fda-warns-cancer-drug-docetaxel-may-cause-symptoms-alcohol. The warning was issued in response to several instances of patients being intoxicated after receiving docetaxel. In two cases doctors decided to use different formulations of the drug with lower alcohol content for subsequent treatments. Sanofi Aventis which markets docetaxel as TAXOTERE revised the package insert by adding alcohol content to the Warnings and Precautions section. Specifically, the package insert refers to the cases of intoxication and warns that the alcohol content in a dose of TAXOTERE is likely to affect the central nervous system and should be controlled for patients in whom alcohol intake should be avoided or minimized.

Due to the known toxicity of ethanol, Layton et al., J. Neurosurgery, 1984, 60 (6), 1134-1137, attempted to reduce the amount of ethanol diluent used for reconstitution of carmustine from 3 mL to 2 mL or 0.75 mL (per 100 mg of carmustine). The amount of carmustine recovered in vitro after simulated clinical administration of the three solutions decreased from 84.9% to 38.3% as the diluent decreased. Clarity and homogeneity decreased as the ethanol content decreased. As an alternative, Layton attempted to dissolve 500 mg of carmustine in 150 mL of 5% dextrose in water. This required 30 minutes of vigorous shaking before there was no visible powder, rendering it impractical for clinical use.

Hence, there is an ongoing need for improved kits which simplify the preparation and improve the safety of injectable carmustine solutions.

### SUMMARY OF THE INVENTION

The present inventors surprisingly found that certain ethanol-free non-aqueous diluents, such as propylene glycol, can be used for reconstitution of lyophilized carmustine. These diluents were found to improve the solubility of carmustine. As a result, a simpler single-step process can be used to prepare injectable carmustine solutions from lyophilized carmustine. Furthermore, a preferred ethanol-free non-aqueous diluent, propylene glycol, was found to result in a reconstituted solution having improved stability compared to reconstitution of carmustine in ethanol.

One embodiment is a kit comprising a product vial containing lyophilized carmustine and a diluent vial containing ethanol-free non-aqueous diluent, preferably propylene glycol. Preferably, the product vial contains only lyophilized carmustine. In one embodiment, the lyophilized carmustine does not contain a bulking agent, such as mannitol. Preferably, the diluent vial only contains the ethanol-free non-aqueous diluent (preferably propylene glycol). The product vial may contain 50-600 mg (e.g., 50-500 mg, 50-200 mg, 300-600 mg, 250-350 mg, or 450-550 mg) of lyophilized carmustine, and the diluent vial may contain 1-18 mL (e.g., 1-15 mL) of the ethanol-free non-aqueous diluent (e.g., propylene glycol). In a preferred embodiment, the product vial contains 100 mg of lyophilized carmustine and the diluent vial contains 3 mL of ethanol-free non-aqueous diluent, preferably propylene glycol (more preferably, sterile propylene glycol). In another preferred embodiment, the product vial contains 50 mg of lyophilized carmustine and the diluent vial contains 1.5 mL of ethanol-free non-aqueous diluent, preferably propylene glycol (more preferably, sterile propylene glycol). In yet another embodiment, the product vial contains 300 mg of lyophilized carmustine and the diluent vial contains 9 mL of ethanol-free non-aqueous diluent, preferably propylene glycol (more preferably, sterile propylene glycol). In yet another embodiment, the product vial contains 500 mg of lyophilized carmustine and the diluent vial contains 15 mL of ethanol-free non-aqueous diluent, preferably propylene glycol (more preferably, sterile propylene glycol). The use of vials containing greater amounts of carmustine and propylene glycol results in fewer opportunities for errors in preparing dilutions, including fewer needle pricks.

Another embodiment is a method of preparing an administrable solution of carmustine comprising (a) dissolving lyophilized carmustine in an ethanol-free, non-aqueous diluent (e.g., propylene glycol) to form a reconstituted solution, and (b) diluting the reconstituted solution with an aqueous 0.9% sodium chloride solution (preferably Sodium Chloride Injection, USP) or an aqueous 5% dextrose solution (preferably 5% Dextrose Injection, USP) to obtain the administrable solution. This method includes a single reconstitution step unlike the current procedure required for BiCNU® which includes two steps to reconstitute the carmustine (i.e., dissolution in 3 mL of ethanol followed by further dissolution in 27 mL of water). The reconstituted solution of the present invention has superior stability compared to reconstitution with 3 mL of ethanol.

In one embodiment, the administrable solution is prepared by (a) dissolving 50-600 mg (e.g., 50-500 mg, 300-500 mg, 300-600 mg, 50 mg, 100 mg, 300 mg, or 500 mg) of lyophilized carmustine in 1-18 mL (e.g., 1-15 mL) (e.g., 3 mL per 100 mg carmustine) of propylene glycol (e.g., sterile propylene glycol or propylene glycol USP) to form a reconstituted solution, and (b) diluting the reconstituted solution with an aqueous 0.9% sodium chloride solution or an aqueous 5% dextrose solution to obtain the administrable solution. In one embodiment, step (a) includes dissolving 300 mg of lyophilized carmustine in 9 mL of propylene glycol. In another embodiment, step (a) includes dissolving 500 mg of lyophilized carmustine in 15 mL of propylene glycol. In yet another embodiment, step (a) includes dissolving 100 mg of lyophilized carmustine in 3 mL of propylene glycol. In yet another embodiment, step (a) includes dissolving 50 mg of lyophilized carmustine in 1.5 mL of propylene glycol. In one embodiment, prior to administration, a total of 600-1200 mg of carmustine is prepared, for example, by separately dissolving at least one 300 or 500 mg of lyophilized carmustine in propylene glycol and 50, 100, 300, or 500 mg of lyophilized carmustine in propylene glycol. Prior to the present invention, when high amounts of carmustine were administered (e.g., at least 300 mg/m² or at least 600 mg), the ethanol diluent necessitated inpatient treatment or an increased post-infusion observation period. With the propylene glycol diluent, carmustine administration even at high doses does not require inpatient treatment or an increased post-infusion observation period (for example, treatment can be performed on an outpatient basis) thereby reducing the cost of treatment.

In a preferred embodiment, the reconstituted solution is stable (≥90% of carmustine remaining) after storage at 2-8° C. for up to 480 or 720 hours or at 25° C.±2° C. for 24 or 48 hours. Step (b), for example, may include diluting the reconstituted solution up to 500 mL with an aqueous 0.9% sodium chloride solution (preferably Sodium Chloride Injection, USP) or an aqueous 5% dextrose solution (preferably 5% Dextrose Injection, USP). Step (b) is preferably performed within 480 hours (or 720 hours) of the reconstituted solution being prepared, where the reconstituted solution is stored at 2-8° C. After storage at 2-8° C. and prior to performing step (b), the reconstituted solution is preferably examined for crystal formation and if crystals are observed, they may be re-dissolved by warming the re-constituted solution to room temperature optionally with agitation. When the reconstituted solution is stored at room temperature, step (b) is preferably performed within 48 hours of the reconstituted solution being prepared. For instance, step (b) may be performed more than 24 hours but less than 480 or 48 hours after the reconstituted solution is prepared. The lyophilized carmustine and the ethanol-free, non-aqueous diluent may be from a kit as described herein. In one preferred embodiment, the administrable solution has a pH in the range of 6 to 7 and an osmolarity in the range of 330-390 mOsmol/L.

Yet another embodiment is a method for administering carmustine to a patient in need thereof by administering by intravenous infusion an administrable solution of carmustine, where the administrable solution is prepared from a kit comprising a product vial containing 100 mg of lyophilized carmustine and a diluent vial containing 3 mL of sterile propylene glycol, and the kit is stored at 2-8° C. The administrable solution is prepared by:
(a) allowing the diluent vial to attain room temperature,
(b) aseptically removing the 3 mL of propylene glycol from the diluent vial (preferably with a below 22 gauge needle), injecting it into the product vial containing 100 mg of lyophilized carmustine, and shaking the product vial to dissolve the lyophilized carmustine to form a reconstituted solution,
(c) optionally, storing the reconstituted solution and prior to performing step (d), the stored reconstituted solution is examined for crystal formation and if crystals are observed, they are re-dissolved by warming the reconstituted solution to room temperature with agitation, and
(d) diluting the reconstituted solution up to 500 mL with an aqueous 0.9% sodium chloride solution or an aqueous 5% dextrose solution to obtain the administrable solution.

Yet another embodiment is a method of administering carmustine comprising intravenously administering an administrable carmustine solution as described herein to a patient in need thereof. The administrable carmustine solution may be prepared as described herein.

Yet another embodiment is a method of treating cancer in a patient in need thereof by intravenously administering an administrable carmustine solution as described herein to the patient. The administrable carmustine solution may be prepared as described herein. The patient may be suffering from brain tumors glioblastoma, brainstem glioma, medulloblastoma, astrocytoma, ependymoma, metastatic brain tumors, multiple myeloma, relapsed or refractory Hodgkin's lymphoma, or relapsed or refractory Non-Hodgkin's lymphomas.

Yet another embodiment is a method for high-dose conditioning treatment of a patient with carmustine comprising administering to the patient an administrable carmustine solution as described herein to the patient with at least one other chemotherapeutic agent. The administrable carmustine solution may be prepared as described herein.

In one embodiment, the method comprises administering the carmustine as part of a BEAM regimen, that is, carmustine (referred to based on the trademark BiCNU in the acronym BEAM) is administered with Etoposide, Ara-C(cytarabine) and Melphalan. For instance, one regimen can include administering 300 mg/m² carmustine on day −6 (six days prior to stem cell transplantation (SCT)), 100 or 150 mg/m² etoposide intravenously Q12H (every 12 hours) on day −5 to −2 (8 total doses), and 200 mg/m² cytarabine intravenously Q12H on days −5 to −2 (8 total doses) and 140 mg/m² melphalan on day −1. Stem cells are administered on day 0.

In another embodiment, the method comprises administering the carmustine as part of a CBV regimen, that is, Cyclophosphamide, carmustine (referred to based on the trademark BiCNU in the acronym CBV) and etoposide (referred to based on its name VP-16). For instance, one regimen can include administering 450-600 mg/m² carmustine on day −7, 900-1600 mg/m² etoposide on day −6 to −4 (Q12H) and 1.8 g/m² cyclophosphamide on day −3 to −2. Stem cells are administered on day 0.

In yet another embodiment, the method comprises administering the carmustine as part of a BEAC regimen, that is carmustine (referred to based on the trademark BiCNU in the acronym BEAM) is administered with Etoposide, Ara-C(cytarabine) and Cyclophosphamide. For instance, one regimen can include administering 300 mg/m² carmustine on day −5, 800 mg/m² etoposide on day −4 to −2 (Q12H), 800 mg/m² cytarabine on day −4 to −2 (Q12H, 1600 mg/m²/day) and 140 mg/kg cyclophosphamide on day −6 and −5. Stem cells are administered on day 0.

In yet another embodiment, any of the methods described herein is performed prior to stem cell transplantation (SCT), for example, in a patient with relapsed and/or refractory lymphoma, such as relapsed/refractory (R/R) non-Hodgkin lymphoma (NHL).

Chemotherapeutic agents include, but are not limited to, alkylating agents (including, but not limited to, cyclophosphamide, ifosfamide, busulfan, chlorambucil, melphalan, temozolomide, cisplatin, carboplatin, and oxaliplatin), topoisomerase I inhibitors (such as irinotecan and topotecan), topoisomerase II inhibitors (such as etoposide, teniposide, doxorubicin, daunorubicin, and idarubicin), mitotic inhibitors (such as vincristine, vinblastine, and taxanes (e.g., docetaxel and paclitaxel)), antifolates (such as methotrexate and pemetrexed), pyrimidine antagonists (such as cytarabine, 5-fluorouruacil, gemcitabine, and capecitabine), purine analogs (such as 6-mercaptopurine (6-MP), azathioprine (prodrug for 6-MP), and cladribine), purine antagonists (such as fludarabine), ribonucleotide reductase inhibitors (such as hydroxyurea (hydroxycarbamide)), bleomycin, actinomycin D, mitomycin, L-asparaginase, proteasome inhibitors (such as bortezomib), and tyrosine kinase inhibitors (such as imatinib and erlotinib).

### DETAILED DESCRIPTION OF THE INVENTION

The terms “ethanol” and “dehydrated alcohol” are used synonymously throughout the specification.

The U.S. Pharmacopeia, USP 42-NF 37 (2019) is hereby incorporated by reference, including the entries for Sodium Chloride Injection, USP, 5% Dextrose Injection, USP.

### Kit

One embodiment is a kit comprising a product vial containing lyophilized carmustine and a diluent vial containing ethanol-free non-aqueous diluent. Preferably, the product vial contains only lyophilized carmustine. In one embodiment, the lyophilized carmustine does not contain a bulking agent. The amount of lyophilized carmustine in product vial may vary from about 2 mg/vial to about 500 mg/vial, preferably 50 mg/vial, 100 mg/vial, 300 mg/vial, and 500 mg/vial. The lyophilized carmustine, which typically is in the form of a powder, may be prepared by methods known in the art, such as those described in U.S. Patent Publication No. 2016/0136116, which is incorporated by reference.

Preferably, the diluent vial only contains the ethanol-free non-aqueous diluent (preferably propylene glycol). Suitable ethanol free non-aqueous diluents include, but are not limited to, aliphatic amides (such as N,N-dimethylacetamide and N-hydroxy-2-ethyl-lactamide), glycols and polyalcohols (such as propylene glycol and glycerine), esters of polyalcohols (such as diacetine (glyceryl diacetate), triacetine (glyceryl triacetate)), polyglycols and polyethers (such as propylene glycol methyl ethers), transcutol, dioxolanes (such as isopropylidene glycerine), N-methylpyrrolidone, or any combination of any of the foregoing. According to one preferred embodiment, the ethanol-free non-aqueous diluent is propylene glycol, N,N-dimethylacetamide, transcutol, or methylpyrrolidone. The ethanol-free non-aqueous diluent is preferably sterile. A more preferred ethanol-free non-aqueous diluent is propylene glycol, such as sterile propylene glycol or propylene glycol USP.

The amount of ethanol-free non-aqueous diluent in the diluent vial may vary from between 1 ml and 20 ml. Preferably the amount of non-aqueous diluent is 1 ml, 2 ml, 3 ml, 4 ml, 5 ml, 6 ml, 7 ml, 8 ml, 9 ml, 10 ml, 11 ml, 12 ml, 13 ml, 14 ml or 15 ml, and more preferably is 3 ml.

In one embodiment, the product vial contains 50-600 mg (e.g., 50-500 mg) of lyophilized carmustine, and the diluent vial contains 1-18 mL (e.g., 1-15 mL) (e.g., 3 mL per 100 mg of carmustine) of the ethanol-free non-aqueous diluent (e.g., propylene glycol). In a preferred embodiment, the product vial contains 100 mg of lyophilized carmustine and the diluent vial contains 3 mL of ethanol-free non-aqueous diluent, preferably propylene glycol (more preferably, sterile propylene glycol or propylene glycol USP).

The vials are preferably made of glass or polypropylene (such as polypropylene which is polyvinyl chloride (PVC) free and di-2-ethylhexyl phthalate (DEHP) free). The vials are preferably not made of (and do not contain) polyvinyl chloride.

In a preferred embodiment, the product vial is stored at 2-8° C. In another preferred embodiment, the product vial and diluent vial are stored at 2-8° C.

### Preparation

In another embodiment, the present invention provides a single-step reconstitution procedure for carmustine injection wherein the lyophilized carmustine, such as from the product vial, is reconstituted with a specified amount of the ethanol-free non-aqueous diluent, such as from the diluent vial.

This reconstitution procedure of the present invention is advantageous over the current procedure used for BiCNU® as it requires a single-step dilution with an ethanol-free non-aqueous diluent. In other words, the additional step of dilution with 27 mL of water for injection as described in the current package insert for BiCNU® is eliminated.

Prior to reconstitution, the diluent vial may be allowed to attain room temperature, for example, by removal from a refrigerator (where it is stored at 2-8° C.). In one embodiment, both the product vial and diluent vial are removed from a refrigerator (where they are stored at 2-8° C.) and allowed to attain room temperature. In one embodiment, the propylene glycol is removed from the diluent vial using an appropriate needle (e.g., a 22 gauge needle or a needle below 22 gauge). In one preferred embodiment, the needle is below 22 gauge. In one embodiment, the propylene glycol is aseptically removed from the diluent vial with a sterile syringe and injected into the product vial containing carmustine. The product vial may be gently shaken to dissolve the carmustine.

The typical two-step reconstitution procedure for the current BiCNU® product (as per its package insert) is as described below:
1) Aseptically removing 3 mL of ethanol diluent from the diluent vial using a sterile syringe and injecting it into the product vial containing the lyophilized carmustine, followed by gentle shaking to obtain a clear solution, and
2) Aseptically adding 27 mL of sterile water for injection into the solution of step (1), followed by gentle shaking to obtain a clear solution.

The single-step reconstitution procedure of the present invention, in contrast, can be as described below:
1) Aseptically removing an appropriate quantity (e.g. 3 mL) of ethanol-free non-aqueous diluent (e.g., propylene glycol) from the diluent vial using a sterile syringe and injecting it into the product vial containing the lyophilized carmustine, followed by gentle shaking to obtain a clear solution.

Preferably, the diluent (preferably propylene glycol) is allowed to attain room temperature before it is aseptically removed from its vial and injected into the product vial. Preferably, the lyophilized carmustine dissolves in the propylene glycol within 3 minutes and more preferably within 2 minutes.

In one embodiment, the reconstituted carmustine solution has a concentration of about 33.3 mg/mL of carmustine.

Prior to administration, the reconstituted carmustine solution may be further admixed with 0.9% sodium chloride injection or 5% dextrose injection to form an administrable solution. For instance, in one embodiment, the reconstituted carmustine solution is further diluted up to 500 mL with 0.9% sodium chloride injection or 5% dextrose injection.

The reconstituted carmustine solution may be stored at room temperature or at 2-8° C. prior to being admixed with the 0.9% sodium chloride injection or 5% dextrose injection. The admixing step is preferably performed within 480 hours of the reconstituted solution being prepared, where the reconstituted solution is stored at 2-8° C. After storage at 2-8° C. and prior to being admixed, the reconstituted solution is preferably examined for crystal formation and if crystals are observed, they may be re-dissolved by warming the re-constituted solution to room temperature optionally with agitation. When the reconstituted solution is stored at room temperature, the admixing step is preferably performed within 48 hours of the reconstituted solution being prepared. For instance, the admixing step may be performed more than 24 hours but less than 480 or 48 hours after the reconstituted solution is prepared.

The reconstituted solution and/or administrable solution may be stored in a glass or polypropylene container (such as a polypropylene container which is polyvinyl chloride (PVC) free and di-2-ethylhexyl phthalate (DEHP) free). These solutions are preferably not stored in a polyvinyl chloride container.

The administrable solution may be a faint yellow colour with a pH in the range of 6 to 7 and osmolarity in the range of 330-390 mOsmol/L. In a preferred embodiment, the administrable solution has a pH of 6.5 and osmolarity of 350-380 mOsmol/L.

The administrable carmustine solution can have a concentration of about 0.2 mg/ml carmustine.

In another embodiment, the reconstituted carmustine solution has improved stability over the reconstituted carmustine solution of the reference product.

As used herein, a “stable” reconstituted carmustine solution means no aggregation was observed when stored at 2 to 8° C. (long-term storage condition) and 25° C.±2° C. (accelerated storage condition) for an appropriate time and where the assay of carmustine is ≥90%.

The carmustine content after storage is determined by high performance liquid chromatography (HPLC method). HPLC was used for performing the assay studies described in the examples below.

Based on the results of Tables 3 and 4 below, it was concluded that the reconstituted carmustine solution of the present invention was stable for up to 720 hours (e.g., for up to 480 hours) when stored at 2° C.-8° C. and for up to 48 hours when stored at 25° C.±2° C. In contrast, the reconstituted carmustine solution of the reference product was stable only under refrigerated conditions (2° C.-8° C.) for up to 96 hours. Accordingly, propylene glycol was found to be superior as a diluent over the dehydrated alcohol diluent of the reference product.

The stability of the admixed carmustine solution was also performed separately at 2° C. to 8° C. (long-term storage condition) for an appropriate time, 25° C.±2° C. (accelerated storage condition) for appropriate time and 2° C. to 8° C. for appropriate time followed by 25° C.±2° C. for appropriate time.

Based on the results of Tables 5 and 6, it was concluded that the admixed carmustine solution of the present invention was stable for 48 hours (2-8° C.)+6 hours (25° C.±2° C.).

### Administration

The carmustine administrable solution may be administered to a patient (e.g., a human patient) by slow intravenous infusion over at least two hours. In one embodiment, the injected area is monitored during the administration. In another embodiment, the rate of administration of the intravenous infusion is no more than 1.66 mg/m²/min. The patient may suffer from cancer.

In one embodiment, the carmustine administrable solution may be administered to a patient to treat brain tumors glioblastoma, brainstem glioma, medulloblastoma, astrocytoma, ependymoma, metastatic brain tumors, multiple myeloma, relapsed or refractory Hodgkin's lymphoma, or relapsed or refractory Non-Hodgkin's lymphomas.

In one embodiment, the carmustine administrable solution is administered to a patient as a single agent or in a combination therapy (such as with other chemotherapeutic agents) to treat (i) brain tumors glioblastoma, brainstem glioma, medulloblastoma, astrocytoma, ependymoma, or metastatic brain tumors, (ii) multiple myeloma in combination with prednisone, (iii) relapsed or refractory Hodgkin's lymphoma in combination with other approved drugs (such as chemotherapeutic agents), or (iv) relapsed or refractory Non-Hodgkin's lymphomas in combination with other approved drugs (such as chemotherapeutic agents).

The carmustine administrable solution may be administered as a single agent in previously untreated patients at a dose of 150 to 200 mg/m² carmustine intravenously every 6 weeks. The carmustine administrable solution may be administered as a single dose or divided into daily injections such as 75 to 100 mg/m² on two successive days. The dose may be lowered when the carmustine administrable solution is used with other myelosuppressive drugs or in patients in whom bone marrow reserve is depleted. The carmustine administrable solution may be administered for the duration according to the established regimen. In one embodiment, the patient is premedicated before each dose with antiemetics.

The dosing (after the initial dose) may be adjusted according to the hematologic response of the patient to the preceding dose. In one embodiment, the patient is dosed as follows:

| | |
|---|---|
| Nadir After Prior Dose | Percentage of Prior Dose to |

| Leukocytes/mm³ | Platelets/mm³ | be Given |
|---|---|---|
| >4000 | >100,000 | 100% |
| 3000-3999 | 75,000-99,999 | 100% |
| 2000-2999 | 25,000-74,999 | 70% |
| <2000 | <25,000 | 50% |

The hematologic toxicity can be delayed and cumulative. In one embodiment, the patient's blood counts are monitored weekly. In another embodiment, a repeat course of the carmustine administrable solution is not administered until circulating blood elements have returned to acceptable levels (platelets above 100 Gi/L, leukocytes above 4 Gi/L and absolute neutrophil count above 1 Gi/L). In yet another embodiment, the interval between courses is 6 weeks.

In one embodiment, the carmustine administrable solution is administered in a high-dose conditioning treatment. One embodiment is a method for high-dose conditioning treatment of a patient with carmustine comprising administering to the patient an administrable carmustine solution as described herein to the patient with at least one other chemotherapeutic agent. The administrable carmustine solution may be prepared as described herein. In one embodiment, the method comprises administering the carmustine as part of a BEAM regimen, that is, carmustine (referred to based on the trademark BiCNU in the acronym BEAM) is administered with Etoposide, Ara-C(cytarabine) and Melphalan. In another embodiment, the method comprises administering the carmustine as part of a CBV regimen, that is, Cyclophosphamide, carmustine (referred to based on the trademark BiCNU in the acronym CBV) and etoposide (referred to based on its name VP-16). In yet another embodiment, the method comprises administering the carmustine as part of a BEAC regimen, that is carmustine (referred to based on the trademark BiCNU in the acronym BEAM) is administered with Etoposide, Ara-C (cytarabine) and Cyclophosphamide. In yet another embodiment, any of the methods described herein is performed prior to stem cell transplantation (SCT) (e.g., hematopoietic stem cell transplantation or autologous stem cell transplantation), for example, in a patient with relapsed and/or refractory lymphoma, such as relapsed/refractory (R/R) non-Hodgkin lymphoma (NHL).

In yet another embodiment, renal function is evaluated prior to administration and/or periodically during treatment. In one embodiment, carmustine treatment is discontinued if the creatinine clearance is less than 10 mL/min. In another embodiment, carmustine is not administered to patients with compromised renal function. In yet another embodiment, transaminases and bilirubin are monitored periodically during treatment.

The following examples further illustrate the invention but should not be construed as in any way limiting its scope. In particular, the processing conditions are merely exemplary and can be varied by one of ordinary skill in the art.

### EXAMPLES

In the examples and tables below, the following terms and abbreviations have the specified definitions.

“IA” refers to Impurity A.

“Impurity A” refers to 1,3-bis(2-chloroethyl) urea.

“IUUI” refers to an individual unspecified unidentified impurity.

“TI” refers to total impurities.

The content of carmustine and impurities was determined by high performance liquid chromatography (HPLC).

### Comparative Example 1 (Reference Product)

TABLE 1
Composition of BiCNU ® (Reference product)

| | |
|---|---|
| Composition | Quantity |

Product Vial

| | | |
|---|---|---|
| Lyophilized Carmustine | 100 | mg |

Diluent Vial

| | | |
|---|---|---|
| Sterile Dehydrated Alcohol (DHA) | 3.0 | ml |

The components in Table 1 above were used to prepare a reconstituted carmustine solution. The reconstitution procedure as described below was followed:
1) Aseptically removing 3 mL of ethanol diluent from the diluent vial using a sterile syringe and injecting it into the product vial containing the lyophilized carmustine, followed by gentle shaking to obtain a clear solution, and
2) Aseptically adding 27 mL of sterile water for injection into the solution of step (1), followed by gentle shaking to obtain a clear solution.

The stability of the reconstituted carmustine solution at 2-8° C. for 720 hours and at 25±2° C. for 120 hours was evaluated. The results are provided in Tables 3 and 4.

### Test Example 1

TABLE 2
Composition of carmustine for injection

| | |
|---|---|
| Composition | Quantity |

Product Vial

| | | |
|---|---|---|
| Lyophilized Carmustine | 100 | mg |

Diluent Vial

| | | |
|---|---|---|
| Sterile Propylene Glycol (PG) | 3.0 | ml |

The product vial and the diluent vial were removed from a refrigerator and allowed to attain room temperature. The single-step reconstitution procedure was performed as follows. 3 mL of propylene glycol was aseptically removed from the diluent vial using a sterile syringe and injected into the product vial containing lyophilized carmustine. The product vial was gently shaken to form a clear solution. The stability of the reconstituted carmustine solution at 2-8° C. for 720 hours and at 25±2° C. for 120 hours was evaluated. The results are provided in Tables 3 and 4.

TABLE 3
Stability data of reconstituted carmustine solution with propylene glycol
(PG) and dehydrated alcohol (DHA) stored at 2° C.-8° C.

| | | | Related Substance (%) | | | | | |
|---|---|---|---|---|---|---|---|---|
| Sampling | | | PG | | | DHA | | |
| intervals | Assay (%) | | Impurity | | Total | Impurity | | Total |
| (Hrs.) | PG | DHA | A | IUUI | Impurities | A | IUUI | Impurities |
| 0 | 99.8 | 98.2 | 0.20 | BLQ | 0.20 | 0.20 | 0.018 | 0.22 |
| 12 | 100 | 96.8 | 0.18 | 0.023 | 0.21 | 0.20 | 0.02 | 0.24 |
| 24 | 101.4 | 96.4 | 0.18 | ND | 0.18 | 0.22 | 0.038 | 0.28 |
| 48 | 101.9 | 94.0 | 0.17 | BLQ | 0.17 | 0.18 | 0.090 | 0.30 |
| 96 | 100.7 | 90.1 | 0.19 | 0.004 | 0.20 | 0.17 | 0.176 | 0.38 |
| 144 | 100.9 | 85.6 | 0.27 | 0.005 | 0.28 | 0.23 | 0.204 | 0.46 |
| 192 | 99.7 | 80.2 | 0.34 | 0.006 | 0.34 | 0.25 | 0.239 | 0.52 |
| 240 | 98.2 | 78.3 | 0.34 | 0.016 | 0.40 | 0.23 | 0.284 | 0.56 |
| 480 | 99.1 | 63.2 | 0.82 | 0.008 | 0.83 | 0.41 | 0.302 | 0.78 |
| 720 | 96 | 51.1 | 1.54 | 0.011 | 1.57 | 0.61 | 0.285 | 0.97 |

TABLE 4
Stability data of reconstituted carmustine solution with propylene glycol (PG)
and dehydrated alcohol (DHA) stored at 25° C. ± 2° C.

| | | | Related Substance (%) | | | | | |
|---|---|---|---|---|---|---|---|---|
| Sampling | | | PG | | | DHA | | |
| intervals | Assay (%) | | | | Total | | | Total |
| (Hrs.) | PG | DHA | Impurity A | IUUI | Impurities | Impurity A | IUUI | Impurities |
| 0 | 99.8 | 98.2 | 0.20 | BLQ | 0.20 | 0.20 | 0.018 | 0.22 |
| 12 | 97.9 | 80.8 | 0.27 | 0.010 | 0.28 | 0.22 | 0.215 | 0.48 |
| 24 | 95.5 | 65.4 | 0.48 | 0.004 | 0.48 | 0.27 | 0.299 | 0.63 |
| 48 | 94.3 | 45.9 | 1.41 | 0.005 | 1.42 | 0.41 | 0.368 | 0.86 |
| 96 | 87.0 | 20.1 | 3.86 | 0.031 | 3.89 | 0.41 | 0.269 | 0.92 |
| 120 | 82.9 | 13.4 | 6.39 | 0.009 | 6.41 | 0.41 | 0.151 | 0.67 |

As shown by Tables 3 and 4, Test Example 1 was stable (≥90% carmustine remaining) for 720 hours when stored at 2° C.-8° C. and for up to 48 hours when stored at 25° C.±2° C. In contrast, the Reference Product was stable (≥90% carmustine remaining) only under refrigerated conditions (2° C.-8° C.) for up to 96 hours.

The reconstituted carmustine solution of Test Example 1 was further admixed with 500 mL of 0.9% sodium chloride injection or 5% dextrose injection to form an admixed carmustine solution for clinical use. The stability of the admixed carmustine solution at (i) 2-8° C. for 24 hours followed by 25±2° C. for 12 hours, (ii) 2-8° C. for 48 hours followed by 25±2° C. for 12 hours, and (iii) 25±2° C. for 8 hours was evaluated. The results are provided in Tables 5 and 6.

TABLE 5
Assay of admixed carmustine solution with 0.9% sodium
chloride injection (NaCl) and 5% dextrose injection

| | Assay (%) | | | |
|---|---|---|---|---|
| Sampling intervals & | PG | | DHA | |
| Storage condition | NaCl | Dextrose | NaCl | Dextrose |

Stored up to 24 hours at 2° C. −8° C. and further
up to 12 hours at 25° C. ± 2° C.

| | | | | |
|---|---|---|---|---|
| Initial (0 hour) | 102.9 | 100.7 | 103.7 | 100.6 |
| 12 hrs. (2-8° C.) | 100.7 | 99.1 | 101.4 | 99.1 |
| 24 hrs. (2-8° C.) | 99.0 | 98.9 | 99.8 | 98.7 |
| 24 hrs. (2-8° C.) + | 94.3 | 94.5 | 94.7 | 94.2 |
| 6 hrs. (25° C. ± 2° C.) | | | | |
| 24 hrs. (2-8° C.) + | 87.3 | 88.1 | 87.2 | 88.0 |
| 12 hrs. (25° C. ± 2° C.) | | | | |

Stored up to 48 hours at 2° C.-8° C. and further
up to 12 hours at 25° C. ± 2° C.

| | | | | |
|---|---|---|---|---|
| Initial (0 hour) | 103.4 | 104.4 | 104.4 | 102.8 |
| 48 hrs. (2-8° C.) | 97.2 | 97.8 | 97.5 | 96.7 |
| 48 hrs. (2-8° C.) + | 92.9 | 93.3 | 92.7 | 92.1 |
| 6 hrs. (25° C. ± 2° C.) | | | | |
| 48 hrs. (2-8° C.) + | 84.9 | 88.0 | 84.4 | 84.9 |
| 12 hrs. (25° C. ± 2° C.) | | | | |

Stored up to 8 hours at 25° C. ± 2° C.

| | | | | |
|---|---|---|---|---|
| Initial (0 hour) | 103.4 | 103.9 | 104.0 | 101.8 |
| 4 hrs. (25° C. ± 2° C.) | 96.2 | 99.1 | 97.2 | 96.3 |
| 8 hrs. (25 ± 2° C.) | 86.4 | 92.5 | 88.7 | 90.9 |

TABLE 6
Impurity data of admixed carmustine solution with 0.9% sodium
chloride injection (NaCl) and 5% dextrose injection

| Sampling | Related Substance (%) | | | | | | | | | | | |
|---|---|---|---|---|---|---|---|---|---|---|---|---|
| intervals and | PG | | | | | | DHA | | | | | |
| storage | NaCl | | | Dextrose | | | NaCl | | | Dextrose | | |
| condition | IA | IUUI | TI | IA | IUUI | TI | IA | IUUI | TI | IA | IUUI | TI |

Stored at 24 Hrs. at 2° C.-8° C. and further for 12 hrs. at 25° C. ± 2° C.

| | | | | | | | | | | | | |
|---|---|---|---|---|---|---|---|---|---|---|---|---|
| Initial (0) | 0.21 | 0.007 | 0.22 | 0.15 | 0.012 | 0.18 | 0.09 | 0.008 | 0.10 | 0.17 | 0.021 | 0.19 |
| 12 hrs (2-8° C.) | 0.25 | 0.009 | 0.27 | 0.22 | 0.022 | 0.28 | 0.16 | 0.010 | 0.17 | 0.18 | 0.031 | 0.22 |
| 24 hrs (2-8° C.) | 0.30 | 0.009 | 0.33 | 0.21 | 0.036 | 0.27 | 0.21 | 0.019 | 0.26 | 0.20 | 0.044 | 0.25 |
| 24 hrs (2-8° C.) + | 0.10 | 0.016 | 0.15 | 0.14 | 0.063 | 0.28 | 0.18 | 0.012 | 0.21 | 0.18 | 0.079 | 0.28 |
| 6 hrs (25° C. ± 2° C.) | | | | | | | | | | | | |
| 24 hrs (2-8° C.) + | 0.15 | 0.018 | 0.20 | 0.14 | 0.108 | 0.31 | 0.13 | 0.012 | 0.17 | 0.16 | 0.127 | 0.38 |
| 12 hrs (25° C. ± 2° C.) | | | | | | | | | | | | |

Stored at 48 Hrs. at 2° C.-8° C. and further for 12 hrs. at 25° C. ± 2° C.

| | | | | | | | | | | | | |
|---|---|---|---|---|---|---|---|---|---|---|---|---|
| Initial (0 hour) | 0.21 | ND | 0.21 | 0.16 | 0.025 | 0.21 | 0.15 | 0.009 | 0.16 | 0.22 | 0.030 | 0.25 |
| 48 hrs (2-8° C.) | 0.23 | 0.032 | 0.32 | 0.19 | 0.101 | 0.36 | 0.16 | 0.012 | 0.20 | 0.18 | 0.123 | 0.34 |
| 48 hrs (2-8° C.) + | 0.14 | 0.027 | 0.20 | 0.16 | 0.142 | 0.39 | 0.18 | 0.015 | 0.22 | 0.17 | 0.174 | 0.40 |
| 6 hrs (25° C. ± 2° C.) | | | | | | | | | | | | |
| 48 hrs (2-8° C.) + | 0.18 | 0.025 | 0.25 | 0.15 | 0.208 | 0.51 | 0.19 | 0.017 | 0.28 | 0.17 | 0.250 | 0.53 |
| 12 hrs (25° C. ± 2° C.) | | | | | | | | | | | | |

Stored at 8 hrs. at 25° C. ± 2° C.

| | | | | | | | | | | | | |
|---|---|---|---|---|---|---|---|---|---|---|---|---|
| Initial (0 hour) | 0.27 | 0.028 | 0.33 | 0.15 | ND | 0.15 | 0.14 | 0.007 | 0.16 | ND | 0.007 | 0.01 |
| 4 hrs. (25° C. ± 2° C.) | 0.27 | 0.013 | 0.30 | 0.20 | 0.047 | 0.29 | 0.16 | 0.014 | 0.18 | 0.26 | 0.030 | 0.38 |
| 8 hrs. (25° C. ± 2° C.) | 0.25 | 0.037 | 0.34 | 0.23 | 0.085 | 0.37 | 0.21 | 0.012 | 0.24 | 0.25 | 0.092 | 0.38 |

As shown by the results in Tables 5 and 6, the admixed carmustine solution of Test Example 1 is stable (≥90% carmustine remaining) for 48 hours (2° C.-8° C.)+6 hours (25° C.±2° C.).

All patents and other references cited herein are hereby incorporated by reference in their entireties.

1. A method for administering carmustine to a patient in need thereof comprising administering by intravenous infusion an administrable solution of carmustine, wherein the administrable solution is prepared by:
(a) dissolving 300 mg to 500 mg of lyophilized carmustine in sterile propylene glycol to form a reconstituted solution, wherein the amount of propylene glycol is 3 mL per 100 mg of carmustine; and
(b) diluting the reconstituted solution with an aqueous 0.9% sodium chloride solution or an aqueous 5% dextrose solution to obtain the administrable solution.

2. The method of claim 1, wherein prior to dissolving the lyophilized carmustine in the propylene glycol, (i) the lyophilized carmustine and propylene glycol are stored in separate vials at 2-8° C. and (ii) the propylene glycol is allowed to attain room temperature just prior to dissolving the lyophilized carmustine in the propylene glycol.

3. The method of claim 1, wherein prior to dissolving the lyophilized carmustine in the propylene glycol, (i) the lyophilized carmustine and propylene glycol are stored in separate vials at 2-8° C. and (ii) the vials are allowed to attain room temperature just prior to dissolving the lyophilized carmustine in the propylene glycol.

4. The method of claim 2 or 3, wherein the propylene glycol is aseptically removed from its vial with a sterile syringe having a needle below 22 gauge and injected into the vial containing the lyophilized carmustine.

5. The method of any one of claims 1-4, wherein the reconstituted solution contains at least 90% of the initial carmustine after storage at 2-8° C. for up to 480 hours.

6. The method of any one of claims 1-5, wherein
the reconstituted solution is stored at 2-8° C. for up to 480 hours prior to step (b), and
after storage at 2-8° C. and prior to performing step (b), the reconstituted solution is examined for crystal formation and if crystals are observed, they are re-dissolved by warming the re-constituted solution to room temperature with agitation.

7. The method of any one of claims 1-6, wherein step (b) comprises diluting the reconstituted solution up to 500 mL with an aqueous 0.9% sodium chloride solution or 500 mL of an aqueous 5% dextrose solution to obtain the administrable solution.

8. The method of any one of claims 1-7, wherein step (b) is performed within 48 hours of the reconstituted solution being prepared.

9. The method of any one of claims 1-8, wherein step (b) is performed more than 24 hours but less than 48 hours after the reconstituted solution is prepared.

10. The method of any one of claims 1-9, wherein the administrable solution of carmustine is administered by intravenous infusion over at least two hours.

11. The method of any one of claims 1-10, wherein the concentration of carmustine in the administrable solution is 0.2 mg/mL.

12. The method of any one of claims 1-11, wherein administrable solution has a pH in the range of 6 to 7 and an osmolarity in the range of 330-390 mOsmol/L.

13. A method for administering carmustine to a patient in need thereof comprising administering by intravenous infusion an administrable solution of carmustine, wherein
the administrable solution is prepared from a kit comprising a product vial containing 300 mg to 500 mg of lyophilized carmustine and a diluent vial containing 9-15 mL of sterile propylene glycol, the kit being stored at 2-8° C., and the administrable solution is prepared by:
(a) allowing the diluent vial to attain room temperature,
(b) aseptically removing the propylene glycol from the diluent vial, injecting it into the product vial containing lyophilized carmustine, and shaking the product vial to dissolve the lyophilized carmustine to form a reconstituted solution, wherein the amount of propylene glycol injected in the product vial is 3 mL per 100 mg of carmustine;
(c) optionally, storing the reconstituted solution and prior to performing step (d), the stored reconstituted solution is examined for crystal formation and if crystals are observed, they are re-dissolved by warming the reconstituted solution to room temperature with agitation, and
(d) diluting the reconstituted solution up to 500 mL with an aqueous 0.9% sodium chloride solution or an aqueous 5% dextrose solution to obtain the administrable solution.

14. The method of claim 13, wherein the propylene glycol is removed from the diluent vial in step (b) with a below 22 gauge needle.

15. A kit for preparing an injectable carmustine solution, the kit comprising a product vial containing lyophilized carmustine and a diluent vial containing sterile propylene glycol, wherein, upon dissolving the lyophilized carmustine in the propylene glycol, the resulting solution contains at least 90% of the initial carmustine after storage at 2-8° C. for up to 720 hours or at 25° C.±2° C. for up to 48 hours.

16. The kit according to claim 15, wherein the concentration of lyophilized carmustine in product vial is from about 2 mg/vial to about 500 mg/vial.

17. The kit according to claim 15 or 16, wherein the amount of propylene glycol in the diluent vial is between 1 ml and 20 ml.

18. The kit according to any one of claims 15-17, wherein in a single-step reconstitution procedure, the lyophilized carmustine from the product vial is reconstituted with specified amount of propylene glycol from the diluent vial.

19. The kit according to any one of claims 15-18, wherein the product vial contains 50-500 mg of lyophilized carmustine and the diluent vial contains 1-15 mL of sterile propylene glycol.
